(19) United States
(12) Patent Application Publication
(10) Pub. No.: US 2001/0007754 A1
(43) Pub. Date: Jul. 12, 2001

(54) SMALL MOLECULE DRUG SCREEN

(76) Inventors: TIMOTHY G. PALZKILL, HOUSTON, TX (US); JOSEPH PETROSINO, HOUSTON, TX (US); WANZHI HUANG, HOUSTON, TX (US)

Correspondence Address: DANIEL F PEREZ, GARDERE & WYNNE LLP, 3000 THANKSGIVING TOWER, 1601 ELM STREET, DALLAS, TX, 75201

(21) Appl. No.: 09/318,476

(22) Filed: May 25, 1999

Related U.S. Application Data
(63) Continuation-in-part of application No. 09/160,405, filed on Sep. 25, 1998.

(51) Int. Cl. G01N 33/543

(52) U.S. Cl. 435/7.2

(57) ABSTRACT

A system for identifying novel antimicrobial agents is disclosed that includes the steps of attaching a β-lactamase inhibitor protein target to a solid support, exposing the β-lactamase inhibitor protein target to a β-lactamase inhibitor protein and an analyte, and detecting the effect of the analyte on the binding of the β-lactamase protein to its protein target, wherein a decrease in binding between the β-lactamase protein target and the β-lactamase inhibitor protein indicates that the analyte affects the interaction between β-lactamase inhibitor protein and its protein target.

This application is a Continuation-In-Part application of United States Letter Patent Application Ser. No. 09/160,405, filed in Sep. 25, 1998.

The government owns certain rights in the present invention pursuant to grant number AI32956 from the National Institutes of Health.

### TECHNICAL FIELD OF THE INVENTION

The present invention relates in general to the field of small antimicrobial agents, and more particularly, to the identification, selection and isolation of small molecules or analytes having bacteriocidal activity against strains of bacteria resistant to β-lactam ring containing, and β-lactam ring related, molecules.

### BACKGROUND OF THE INVENTION

Without limiting the scope of the invention, its background is described in connection with small molecular agents or analytes that act to replace or enhance the action of penicillin and cephalosporin related antimicrobial agents, as an example.

Heretofore, in this field, β-lactam antibiotics, such as the penicillins and cephalosporins, have been the most often used antimicrobial agents for the treatment of bacterial infection and infectious disease. Because of their widespread use, bacterial resistance to these antibiotics has become an increasing problem (Davies, J., “Inactivation of Antibiotics and the Dissemination of Resistance Genes,” Science, 264:375-382 (1994)). The most common mechanism of bacterial resistance is the production of β-lactamases that break the β-lactam portion of antibiotics. β-lactamases are generally secreted to the periplasm of gram-negative bacteria (or extracellularly gram-positive bacteria), where they hydrolyze, and thereby inactivate, the β-lactam ring of these antibiotics. There are a large number of β-lactamases that are found encoded either on plasmids or on the bacterial chromosome (Bush, K., G. A. Jacoby, and A. A. Medeiros, “A Functional Classification Scheme for β-lactamases and its Correlation With Molecular Structure,” Antimicrob. Agents Chemother., 39:1211-1233 (1995)). In gram-negative bacteria, for example, the most common plasmid-based β-lactamase is the TEM-1 β-lactamase.

One effective means of combating TEM-1 β-lactamase mediated resistance has been the clinical use of small molecule β-lactamase inhibitors such as sulbactam and clavulanic acid (Parker, R. H., and M. Eggleston, “β-lactamase Inhibitors: Another Approach to Overcoming Antimicrobial Resistance,” Infect. Control., 8:36-40 (1987)). These molecules, however, do not possess significant antimicrobial activity themselves but are used in conjunction with other β-lactam antibiotics, such as ampicillin. This class of molecules act by protecting the antibiotic from the action of 4-lactamase and thereby restore the therapeutic value of the antibiotic agent.

While the combination approach has worked to increase the effectiveness of β-lactam antibiotics for a period of time, this has been only a reprieve. New reports of resistance to β-lactam:β-lactamase inhibitors due to mutations in β-lactamase have enabled bacteria to avoid inactivation by the inhibitor while retaining the ability to hydrolyze β-lactam antibiotics (Imtiaz, U., E. Billings, J. R. Knox, E. K. Manavathu, S. A. Lerner, and S. Mobashery, “Inactivation of Class A β-lactamases by Clavulanic Acid: The Role of Arginine 244 in a Proposed Nonconcerted Sequence of Events,” J. Am. Chem. Soc., 115:4435-4442 (1993)).

The need to identify and isolate novel antimicrobial agents is further accentuated by the identification of mutations within β-lactamase that allow it to hydrolyze antibiotics designed to circumvent the enzyme's primary activity. In some gram-positive bacteria, such as Streptococcus pneumoniae, resistance to β-lactam antibiotics is acquired by mutations in the penicillin-binding-proteins targeted by the drugs. For example, methicillin-resistant Staphylococcus aureus (MRSA) has acquired the penicillin-binding-protein (PBP) PBP2a, which is able to catalyze the cross-linking of the bacterial cell-wall, but does not bind any β-lactam antibiotics. Many MRSAs are also resistant to other classes of antibiotics as well, and as a result, some MRSA infections are only treatable with the glycopeptide antibiotic vancomycin.

One such method of screening is shown in PCT patent application WO 98/19162, of Novalon Pharmaceutical Corporation, filed by Fowlkes, et al. The disclosed method includes the screening of at least one potential surrogate library for members binding to a target protein to find an inhibitory compound; screening at least one complementary library; and determining whether the inhibitory compound mediates biological activity of the target protein. The method disclosed, however, depends on disrupting the interaction between two binding proteins, which may be disrupted for reasons independent of the activity of the inhibitory compound on an active site.

### SUMMARY OF THE INVENTION

It has been found, however, that the present methods for identifying and customizing new antimicrobial agents to drug resistant strains of bacteria are unable to cope with the increase in nosocomial infection that are multiple-drug resistant (MDR). A significant problem of current isolation and identification systems is that they rely on the serendipitous isolation and characterization of antimicrobial agents. Alternatively, rational drug design systems based on the X-ray structure of the target require the structure of the target to be known.

The system disclosed herein has been used to facilitate the screening of novel inhibitors with potent activity for the extended spectrum β-lactamases (ESBLs) and for PBPs, as well as for the interaction between these and other molecules. These same engineered molecules may be used to increase the specificity of the screen for particular PBPs.

The small molecule inhibitor of β-lactamases, clavulanic acid, is a natural product from Streptomyces clavuligerus. In addition to clavulanic acid, S. clavuligerus also produces a protein inhibitor of β-lactamase called β-lactamase inhibitory-protein (BLIP). BLIP is a 165 amino acid protein encoded by the bli gene that binds and inhibits TEM-1 β-lactamase with a reported Kᵢ of 0.6 nM. In addition, BLIP has been reported to inhibit the Enterococcus faecalis PBP5 with a Kᵢ of 12 μM. As BLIP binds to β-lactamases from both gram-negative and gram-positive bacteria (albeit with reduced affinity) the present inventors recognized that BLIP-β-lactamase interactions could be exploited to develop a system to identify, and improve the affinity of, antimicrobial agents.

The X-ray structure of BLIP has been solved both alone and in complex with TEM-1 β-lactamase to reveal the residues making up the binding surface of BLIP (Strynadka, N. C. J., S. E. Jensen, P. M. Alzari, and M. N. G. James, “A Potent New Mode of β-lactamase Inhibition Revealed by the 1.7 Å X-ray Crystallographic Structure of the TEM-1-BLIP Complex,” Nature Struct. Biol. 3:290-297 (1996); and Strynadka, N. C. J., S. E. Jensen, K. Johns, H. Blanchard, M. Page, A. Matagne, J. -M. Frere, and M. N. G. James, “Structural and Kinetic Characterization of a β-lactamase-inhibitor Protein,” Nature, 368:657-660 (1994)). The present inventors realized, however, that the X-ray crystallographic data has failed to lead to rational strategies for drug design, as the TEM-1 β-lactamase has vastly different interaction characteristics from other β-lactamases. Id.

To overcome these and other problems in the art, the present inventors have expressed BLIP in bacteria as a fusion protein with the g3p coat protein of the M13 bacteriophage. Recombinant bacteriophage expressing the fusion protein are able to bind specifically, and with high affinity, to the TEM-1 β-lactamase. Therefore, the BLIP-g3p fusion protein was expressed and displayed on the surface of the bacteriophage in a folded, functional form.

Display of functional BLIP was accomplished by constructing a new phage display vector that allowed the BLIP protein to be expressed under the control of the constitutive TEM-1 β-lactamase promoter and secreted under the direction of the (3-lactamase signal sequence. The low-levels of transcription of the BLIP-g3p fusion are not toxic to E. coli, leading to titers of 1×10¹² to 1×10¹³ phage/ml.

Another problem overcome by the present inventors was the high frequency of frameshift mutations of the BLIP gene found among transformants after insertion of PCR produced BLIP gene into the pG3-C3 vector in order to produce recombinant BLIP. To obtain wild-type transformants it was necessary to obtain a non-mutant sequence by a functional selection for BLIP-phage that bound to immobilized β-lactamase. A high frequency of clones contained frameshift mutations in BLIP in the non-selected population, which may be due to a high frequency of polymerase errors during PCR on templates having a high G-C content.

The development of the BLIP-phage system and the recombinant production of BLIP was then exploited to determine which residues on BLIP are critical for TEM-1 β-lactamase binding and to select for variants that bind tightly to other β-lactamases or penicillin binding proteins. To achieve this, libraries of random mutants of BLIP were created in the pG3-BLIP vector. The phage libraries were then panned on purified TEM-1 β-lactamases and other β-lactamase, as described hereinbelow.

More particularly, one embodiment of the present invention is a system for identifying novel antimicrobial agents including the steps of; attaching a β-lactamase inhibitor protein target to a solid support, exposing the β-lactamase inhibitor protein target to a β-lactamase inhibitor protein and an analyte, and detecting the effect of the analyte on the binding of the β-lactam binding protein target to its protein target, wherein a decrease in binding between the β-lactamase protein and the β-lactamase inhibitor protein as compared to a control sample is indicative that the analyte affects the interaction between a β-lactamase inhibitor protein and its protein target.

The present inventors have used site-directed mutagenesis techniques, and the creation of mutant libraries, to increase BLIP binding and inhibition of recombinant β-lactamases and recombinant penicillin-binding proteins. Using the tools and the system developed by the present inventors, the PBP-BLIP interaction has been used to identify molecules that disrupt the interaction between BLIP and PBP. The same assay may be used to screen known and unknown analytes for new compounds that disrupt the interactions like those of PBP-BLIP. The PBP-BLIP assay disclosed herein may also be used to screen for, and increase the affinity of, BLIP-PBP interaction inhibitors.

One example of potential analytes are small BLIP fragments, and mutants thereof, having reduced antigenicity that have improve access to sites of infectious infection and disease. Other examples of analytes are small molecules that may or may not be derived from presently used antimicrobial agents having non-cleavable variations of β-lactam ring structures. The small molecules may not even be related to biologically compatible molecules, such as left handed amino acids and sugars. In addition, small molecule collections from combinatorial chemistry libraries may be used. The small molecules that inhibit the interaction between β-lactamases and β-lactamase inhibitor proteins may be related to molecules such as clavulanic acid or sulbactam. The present invention is also adaptable to high throughput analysis systems, such as automated ELISA.

Regarding the detection methods of the present invention, the phrase “a decrease in binding of” proteins means that upon exposing the two or more proteins that are generally capable of interacting, for example BLIP and a BLIP target, with an analyte under appropriate conditions of ionic strength, temperature, pH and the like, a decrease in the specific binding will occur. The interaction occurs due to specific electrostatic, hydrophobic, entropic or other interaction of certain amino acid or glycolytic residues of the one protein, BLIP, with specific amino acid or glycolytic residues of the second protein, particularly a BLIP target, to form a stable complex under the conditions effective to promote the interaction. The disruption of the interaction between the protein may alter the three dimensional conformation of either or both proteins or polypeptides involved in the interaction and it may also alter the function or activity of either or both proteins or polypeptides involved in the interaction.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 shows a map of the relative positions and restriction endonuclease cleavage sites of the pG3-BLIP plasmid that encodes a BLIP-g3p fusion protein;

FIGS. 2A and 2B are graphs showing the results of an ELISA assay for phage and the affinity of the BLIP-phage;

FIG. 3 is a drawing of a positive control for a small molecule or analyte screen;

FIG. 4 is a drawing of a screen for a small molecule or analyte;

FIG. 5 is a graph showing the amount of bound BLIP with and without BLIP-like inhibitors; and

FIG. 6 is a graph showing the amount of bound BLIP with and without BLIP-like inhibitors using an M13 phage display construct of BLIP.

### DETAILED DESCRIPTION OF THE INVENTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention and do not delimit the scope of the invention.

The display of proteins on the surface of filamentous phage has been shown to be a powerful method to select variants of a protein with altered binding properties from large combinatorial libraries of mutants. The β-lactamase inhibitory protein (BLIP) is a 165 amino acid protein that binds and inhibits TEM-1 β-lactamase-catalyzed hydrolysis of the penicillin and cephalosporin antibiotics. The present inventors have constructed a new phagemid vector and have developed a method of using the vector as part of a system to display or produce BLIP on the surface of filamentous phage. The recombinant BLIP protein was shown to have binding to immobilized β-lactamase. The binding of the recombinant BLIP to β-lactamase was specific as it can be competed off by the addition of soluble β-lactamase.

A two-step phage ELISA procedure was also used to demonstrate that the BLIP-displaying phage bind β-lactamase with an IC₅₀ of 1 nM, which compares favorably with a previously published K₁ of 0.6 nM. Therefore, the present inventors have developed the tools necessary to accompany a system for protein engineering of BLIP to expand its binding range to other β-lactamases and penicillin binding proteins and for the detection and isolation of small molecule drugs that are bacteriocidal, bacteriostatic or that enhance the ability to control bacterial infection and disease by other molecules, such as antibiotics.

To amplify and select for specific high β-lactamase binders, phage display isolation was used to increase the amount and level of molecular interactions between the potential inhibitor and the β-lactamase. Phage display has proven to be an effective methodology for the selection of binding partners of high affinity or altered specificity.

Briefly, monovalent display of a protein of interest may be achieved by fusing the gene encoding the protein to the N-terminus of the M13 gene III coat protein. In the present invention, high affinity variants are obtained by creating phage libraries containing mutants of the protein of interest and sequencing the corresponding DNA packaged in the phagemid particles after several rounds of binding selection.

The first step in the phage display system is the cloning of the BLIP gene into a new phagemid vector. The vector encodes chloramphenicol resistance and contains an amber codon at the 5′end of gene III. BLIP is fused at its N-terminus to the β-lactamase signal sequence and at its C-terminus to the protein encoded by gene III of the phage (g3p). Transcription of the fusion is controlled by insertion upstream from the fusion protein of the constitutive β-lactamase promoter. Specific binding of phage containing the BLIP-g3p fusion to immobilized β-lactamase is demonstrated to occur with nanomolar affinity, and has been used to engineer new BLIP binding specificities.

Bacterial Strains. Escherichia coli strain XL1-Blue [F′::Tn10proA⁺B⁺lacI^{q} Δ(lacZ) M15/recA1 endA1 gyr96(Nal^{r}) thi hsdR17 (r⁻m⁺) supE44 relAl lac] was used for transformations of ligations and E. coli TG1 [F′ traD36⁺lacI^{q} Δ(lacZ) M15 proA⁺B⁺/supE Δ(hsdM⁻mcrB⁻) (r⁻^{—}McrB⁻) thi Δ(lac⁻proAB) was used for production, amplification, and titering of bacteriophage.

The phagemid encoding the BLIP-g3p fusion was constructed by inserting a 1365 bp XbaI-BamHI fragment containing gene III from phagemid phGHamg3 (Lowman, H. B., S. H. Bass, N. Simpson, and J. A. Wells, “Selecting High-Affinity Binding Proteins by Monovalent Phage Ddisplay,” Biochemistry, 30:10832-10838 (1991)) into XbaI-BamHI digested pBCKS+(Stratagene, U.S.A.) to create plasmid pG3-CMP. The pG3-CMP plasmid was digested with SalI, the 5′overhangs were filled-in with Klenow polymerase and dNTPs, and the ends were religated to destroy the SalI site and create plasmid pG3-C2.

S. clavuligerus genomic DNA was used as template for amplification of the bli gene. S. clavuligerus (ATCC 27064) cultures were grown in TSA broth containing 1% starch for 64 hours. Chromosomal DNA was isolated using the Puregene kit (Gentra) for Gram-positive bacteria. The bli gene was amplified using PCR primers, the 5′ primer BLIPXHOI contains a XhoI site and the 3′ the primer BLIPXBAI contains an XbaI site. The primers were designed to amplify only the mature portion of BLIP which includes codons 37 to 165 of the bli gene (Doran, J. L., B. K. Leskiw, S. Aippersbach, and S. E. Jensen, “Isolation and Characterization of a β-lactamase-Inhibitory Protein from Streptomyces clavuligerus and Cloning and Analysis of the Corresponding Gene,” J. Bacteriol., 172:4909-4918 (1990)). The BLIP PCR product was purified using a QIAquick PCR Purification kit from Qiagen (Qiagen, U.S.A.) following the instructions of the manufacturer. The purified product was digested with XhoI and XbaI and gel-purified on a SeaPlaque low melt agarose gel. The pG3-C3 plasmid was digested with SalI and XbaI to release the β-lactamase gene. The remainder of the vector was purified from the released β-lactamase gene by low melt agarose.

FIG. 1 shows a map of the relative positions and restriction endonuclease cleavage sites of the pG3-BLIP plasmid that encodes and expresses a BLIP-g3p fusion protein of, and for use with, the invention. The XhoI-XbaI digested BLIP fragment is inserted into the SalI-XbaI digested of the pG3-C3 vector, described hereinabove, to create a fusion of the β-lactamase signal sequence fused to the N-terminus of BLIP and g3p at the C-terminus of BLIP. The BLIP-g3p fusion containing pG3-BLIP plasmid was electroporated into E. coli XL1-Blue and individual colonies were screened for BLIP inserts by DNA restriction enzyme analysis.

A 6×Histidine-tag (6×His-tag) was then inserted between the β-lactamase signal sequence and the BLIP coding sequence of pG3-BLIP by overlapping PCR mutagenesis. A SacI site in PD-blal and a Xbal site in MALBLI-2 allowed the PCR product to be cloned into SacI and XbaI digested pG3-BLIP following treatment of the vector with calf-intestinal alkaline phosphatase (CIP). The final SacI/XbaI fragment contains, from 5′to 3′, the β-lactamase constitutive promoter, the β-lactamase periplasmic signal sequence, and a BLIP N-terminus 6×xHis-tag followed by the mature BLIP coding sequence, followed by gene III.

Phage preparation and panning. After overnight growth, E. coli cells were removed by centrifugation and the phage were precipitated from the supernatant with ⅕ volume of 20% PEG, 2.5 M NaCl. The phage were pelleted by centrifugation and resuspended in 1/100of the original culture volume of STE (0.1 M NaCl, 10 mM Tris-Cl pH 8.0, 1 mM EDTA pH 8.0). The phage titer was determined by making serial dilutions of 0.1 ml total volume and adding 0.2 ml of E. Coli TG1 cells. Aliquots of 0.15 ml were plated on LB agar supplemented with 12.5 μg/ml of chloramphenicol. After overnight growth at 37° C., the number of colonies was determined and the titer was calculated.

β-lactamase was immobilized for panning by covalent attachment to 1 μm oxirane-acrylic beads (Sigma, U.S.A.). Briefly, 50 mg of beads was suspended in 0.1 M sodium carbonate buffer (pH 8.6) and was incubated with purified TEM-1 β-lactamase at a concentration of 0.04 mg/ml for 24 hrs at 4° C. Unreacted oxirane groups were blocked by incubating with 10 mg/ml bovine serum albumin (BSA) overnight at 4° C. The beads were then pelleted and washed several times with buffer A which is Tris-buffered-saline containing 1 mg/ml BSA and 0.5 g/l Tween 20. The TEM-1 containing beads were stored in buffer A in a final volume of 0.5 ml.

For panning, 1×10¹¹ phage were contacted with 5 mg of β-lactamase conjugated oxirane beads in a final volume of 0.5 ml in buffer A. The phage-β-lactamase bead mixture was incubated for 2 hrs at room temperature with rocking to reach equilibrium. The phage-β-lactamase beads were then washed 10 times with 0.75 ml of buffer A. The bound phage may be eluted from the phage-β-lactamase beads by incubation with, e.g., 0.2 ml of elution buffer (0.1 M glycine pH 2.2, 1 mg/ml BSA, 0.5 g/l Tween 20, 0.1 M KCl) for 30 minutes. Those of skill in the art will recognize that other elution buffers may be used to release the phage from the β-lactamase beads. Elution buffers may or may not affect phage viability, where they do, the phage DNA may isolated and repackaged into new virions by transformation of E. coli.

The elution mixture may be neutralized with 25 μl of 1 M Tris-Cl pH 8.0. The phage titer of the elution mixture was determined as described above. The eluted phage were amplified by adding 0.15 ml of the neutralized elution mixture to 5 ml of E. coli TG1 cells. After 30 minutes incubation at room temperature, 25 ml of 2YT medium was added along with 1×10⁹ VCS M13 helper phage (Stratagene, U.S.A.). The phage were precipitated as described above after overnight incubation with shaking at 37° C.

Phage ELISA. A two-step phage ELISA was performed to measure BLIP-phage affinity for TEM-1 β-lactamase. Microtiter plates (Nunc, U.S.A., Maxisorp, 96 well) were coated with purified TEM-1 β-lactamase (at 10 μg/ml) in 50 mM sodium carbonate (pH 9.6) at 4° C. overnight. The plates were then blocked with SuperBlock (Pierce) for 2 hours at room temperature. Serial dilutions of the BLIP phage stock were added to the wells and incubated for 2 hours at room temperature in buffer A at a final volume of 0.15 ml. After washing the plates several times with buffer A, the bound phage were probed with a sheep anti-M13 polyclonal antibody conjugated to horseradish peroxidase. To determine phage affinity, serial dilutions of β-lactamase and a subsaturating concentration of 1.3×10¹¹ BLIP phage were added to wells in 0.1 ml of buffer A. After 2 hours at room temperature, the wells were washed multiple times with buffer A and bound phage were probed as described above.

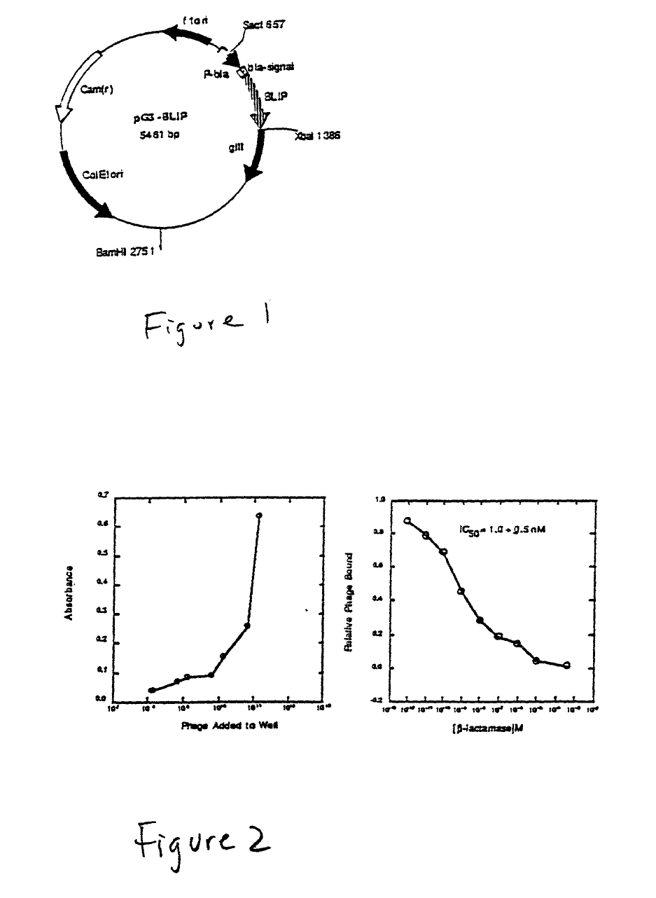

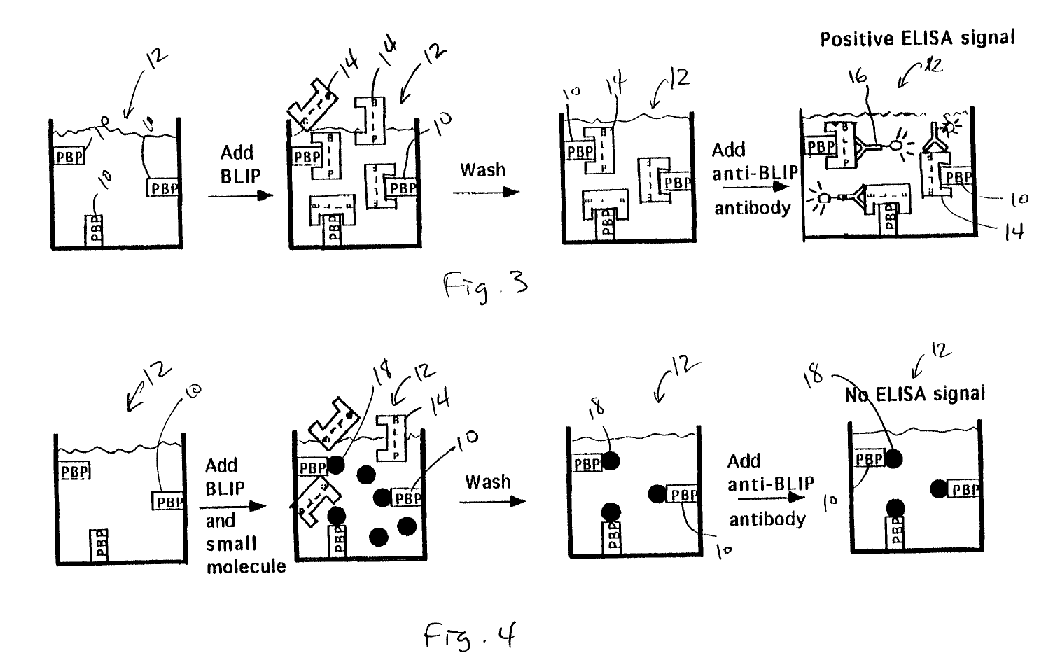

FIG. 2A is a graph of the binding curve used to determine the affinity of BLIP-g3p phage to TEM-1 coated ELISA plates at subsaturating concentration of phage (1.3×10¹¹ phage). The IC₅₀ value given shows the concentration of competing β-lactamase that results in half-maximal binding to the phagemid. FIG. 2B shows the relative optical density of the expressed BLIP-g3p fusion protein on the surface of chimeric phage in the phage ELISA assay. To quantitate binding, β-lactamase was coated onto ELISA plates and a constant subsaturating concentration of pG3-BLIP phage was added with serial dilutions of purified β-lactamase.

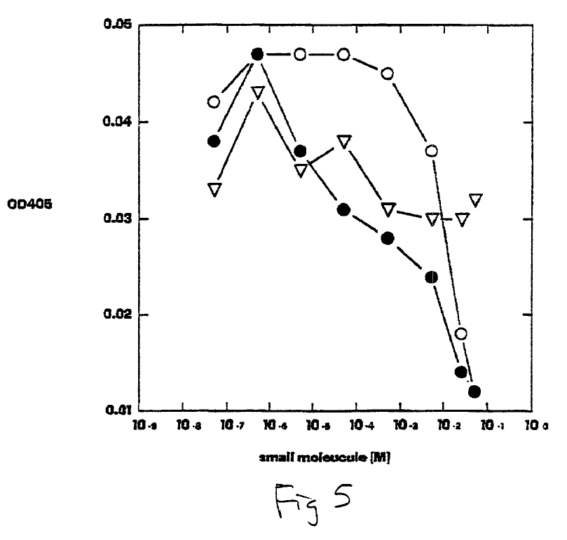

The half-maximal concentration was calculated by converting the data in FIG. 3 from log to linear values and fitting the binding curve to the equation for a hyperbola. Affinities (IC₅₀) were calculated as the concentration of competing β-lactamase that resulted in half-maximal BLIP phage binding. As seen in FIG. 2, the pG3-BLIP phage were competed off of the immobilized β-lactamase with soluble β-lactamase at an IC₅₀ of 1 nM. This affinity compares favorably to the published Kᵢ value of 0.6 nM for the BLIP-β-lactamase interaction. Therefore, the inventors have expressed BLIP on the surface of the bacteriophage in a form that binds tightly and specifically to β-lactamase.

The next step in the antimicrobial isolation system is to specifically enrich for BLIP-phage by panning on β-lactamase. In order to use the pG3-BLIP phagemid to engineer the BLIP protein for altered binding properties it is necessary to be able to select binding phage by panning on an immobilized substrate. BLIP-phage was enriched as demonstrated by attaching purified β-lactamase to oxirane-acrylic beads and incubating the beads with 1×10¹¹ phage from the pG3-BLIP phage stock in the presence of a large excess of BSA.

Two controls were performed to demonstrate that phage binding to the beads was dependent on the BLIP-β-lactamase interaction. First, an internal control was performed by adding 1×10¹¹ phage that did not display BLIP along with the 1×10¹¹ pG3-BLIP phage to the oxirane beads conjugated to β-lactamase. The phagemid, pG3-SPT, which produced the non-displaying phage was constructed by inserting a gene cassette encoding spectinomycin resistance (Reece, K. S., “New Plasmids Carrying Antibiotic-Resistance Cassettes,” Gene, 165:141-142 (1995)) into the chloramphenicol resistance gene of pG3-C2.

One advantage in the design of this antimicrobial agent selection system is that the extent of enrichment of non-displaying phage versus BLIP-displaying phage may be determined simply by titering the phage recovered from the oxirane beads on spectinomycin-containing agar plates as well as chloramphenicol-containing agar plates. The results in Table 1 illustrate that 27-fold more pG3-BLIP phage were recovered from the β-lactamase beads than non-displaying phage when no soluble β-lactamase is added to compete for binding to the pG3-BLIP phage (compare pG3-BLIP versus pG3-SPT in bla beads +0 μM bla column).

TABLE I

| | bla^{a} beads + | bla beads + | bla beads + | bla beads + | |
|---|---|---|---|---|---|
| Phage | 0 μM bla | 0.1 μM bla | 1 μM bla | 10 μM bla | BSA beads |
| pG3-BLIP | 2.3 × 10^{6 b} | 3.4 × 10⁵ | 2.7 × 10⁵ | 1.9 × 10⁵ | 1.8 × 10⁵ |
| pG3-SPT | 8.6 × 10^{4 b} | 9.9 × 10⁴ | 6.9 × 10⁴ | 6.7 × 10⁴ | 8.8 × 10⁴ |

^{a}bla represents β-lactamase
^{b}The number of phage particles eluted from beads after the application of 1 × 10¹¹ phage in the binding reaction. Note that each column represents a binding experiment and in each experiment 1 × 10¹¹ of pG3-BLIP phage and pG3-SPT phage were added simultaneously to the beads.

As increasing amounts of soluble β-lactamase were added the enrichment of pG3-BLIP phage over non-displaying phage decreased to 3-fold when 10 μM soluble β-lactamase was added (bla beads+10.0 μM bla column). Therefore, soluble β-lactamase is able to compete off the binding of pG3-BLIP phage to the β-lactamase coated oxirane beads. These data show that BLIP phage was specifically enriched by a round of panning against immobilized β-lactamase.

For the second control, 1×10¹¹ pG3-BLIP phage along with 1×10¹¹ non-displaying pG3-SPT phage were incubated with oxirane beads to which only BSA had been attached. The data in Table 1 indicate that 13-fold more BLIP-phage were bound to β-lactamase than to the BSA control (compare pG3-BLIP in the bla beads+0 μM bla versus BSA beads columns). In addition, there was only a 2-fold difference in the number of BLIP-phage versus non-displaying phage recovered from the BSA beads (compare pG3-BLIP and pG3-SPT in the BSA beads column). This is in contrast to the 27-fold difference between pG3-BLIP phage and non-displaying phage recovered from β-lactamase beads described above. These data show that BLIP phage bind specifically to immobilized β-lactamase.

Expression of Soluble BLIP. BLIP from Streptomyces clavuligerus ATCC 27064 was cloned into pG3-cmp to form pG3-BLIP. This vector enabled BLIP to be expressed as a fusion to the M13 gene III protein and subsequently displayed on the surface of M13 bacteriophage. The construct is expressed under the constitutive β-lactamase promoter, and also possesses the β-lactamase signal sequence fused to the N-terminus of BLIP. Induction is not necessary for expression, and the fusion protein is transported to the periplasm as evidenced by the proper formation of phage displaying BLIP. In the construction of pGR32, an N-terminal 6×His-tag is inserted between BLIP and the β-lactamase signal sequence from the pG3-BLIP construct. The SacI/XbaI fragment containing the β-lactamase promoter and signal sequence, the 6×His-tag, and the BLIP coding sequence was then cloned into pTP123, as described hereinabove, so that expression may be directed under the β-lactamase constitutive promoter or the IPTG-inducible trc promoter.

E. coli RB791 (Strain W3110 lacI^{qL8})(available from ATCC) was used to express BLIP and the D49A and F142A BLIP mutants. Plasmid pTP123 is a cmp^{r} amp^{r} derivative of pTrc 99A (Pharmacia, Sweden). It was created by ligating the Smal cassette from pKRP10 into BsaI and XmnI digested pTrc 99A. The BsaI and Xmnl sites were filled in using the Klenow fragment of DNA Polymerase I prior to ligation. This cloning step inserts a chloramphenicol acetyl transferase (cat) gene into the rrnBT₁T₂ transcriptional terminators and part of the β-lactamase gene encoded by pTrc 99A. As a result, functional β-lactamase is not expressed, and potential difficulty in BLIP purification due to binding of endogenous β-lactamase is avoided. The cat gene in pTP123 is in the same orientation as the trc promoter.

The sequence of 6×His-tag clone was confirmed by the dideoxy-chain termination method and was named pGR32. The positioning of this construct in pTP123 allows N-terminal 6×His-tag BLIP to be expressed either under the β-lactamase constitutive promoter, or by induction of the trc promoter with IPTG. The 6×His-tag facilitates the purification of BLIP using an appropriate nickel or cobalt based affinity column, while the β-lactamase signal sequence enables BLIP to be transported to the periplasmic space, thus eliminating the need to isolate whole cell extracts for BLIP purification.

Several growth conditions and IPTG concentrations were used for optimal expression of BLIP. It was found that growth at 25° C., and addition of 3 mM IPTG to E. coli RB791 cells harboring the pGR32 plasmid increased expression of BLIP above background levels. The 6×His-tag allows BLIP, as well as the D49A and F142A BLIP mutants, to be purified to greater than 90% homogeneity using a TALON affinity column (Clontech, U.S.A.). Concentrations of BLIP were first determined by the method of Bradford, and then quantitative amino acid analysis was performed to confirm the Bradford results (Protein Core Facility-Baylor College of Medicine).

BLIP and β-lactamase Expression and Purification. The first step toward identifying the amino acids important for BLIP specificity and inhibitory activity was to develop an expression system designed to produce BLIP with wild-type activity. BLIP expressed in its native S. clavuligerus is straightforward and produces large quantities of protein, while expression in another Streptomyces species, S. lividans, produces limited quantities of BLIP. The inventors have now been able to express BLIP in E. coli in order to be able to use protein engineering/selection techniques, such as phage display, to be used. Successful expression of soluble BLIP in E. coli now permits the production, identification and isolation of BLIP mutants. The soluble expressed BLIP and mutants derived as shown herein and using a mutagenesis and selection system as described hereinabove, complements the phage display system by allowing soluble, engineered BLIP mutants to be purified and tested against its target β-lactamase or PBP.

Different methods of expressing BLIP in E. coli, have been attempted with little success. It is not known whether the presence of rare codon, a high GC content (69%), or the requirement for Streptomyces-specific post-translational modification are responsible for this difficulty. An additional possibility is that BLIP itself is toxic to E. coli. Small quantities of BLIP displaying wild-type inhibitory kinetics, however, were purified using a maltose binding protein fusion system. Optimization of this fusion system was difficult as much protein was lost in the additional purification steps needed to obtain pure 6×His-tagged BLIP proteins were purified in a relatively simple manner, while usually maintaining the native activity of the tagged-protein. Therefore, an expression system centered around an N-terminal 6×His-tag BLIP was used. Expression is directed by the inducible trc promoter, and a cat gene is inserted into the plasmid's β-lactamase gene to avoid possible complex formation during purification of BLIP. This system enabled BLIP to be purified to 90% homogeneity in one step.

Plasmid pGR32, pJP128, and pJP129 were transformed into E. coli RB791 by electroporation. An overnight culture of each was grown shaking in 40 mL Luria-Bertani (LB) medium at 37 C. in the presence of 12.5 μg/mL chloramphenicol. The 40 ml of overnight culture were used to inoculate 2 L of LB media containing 12.5 μg/mL chloramphenicol. The bacteria then grown shaking at 25 C. until OD₆₀₀=1.2. For induction of BLIP, 3 mM IPTG was added to each culture, and the cultures were then allowed to grow an additional 5 hours.

Following the 5 hour induction, the cells were pelleted and resuspended in 15 mL sonication buffer (20 mM Tris-HCl (pH 8.0) and 500 mM NaCl). The cells were then sonicated in two batches, and insoluble material was pelleted by centrifugation. The soluble protein in the supernatant was purified over a 4 mL TALON column (Clontech) according to the manufacturer's instructions. A 4 mM imidizole wash step was utilized to remove protein from the column which bound less tightly than the 6×His-tagged BLIP. BLIP was eluted using an elution buffer consisting of 50 mM imidizole added to the sonication buffer (pH 8.0). Fractions were examined by SDS-PAGE to estimate purity and yield. Approximately 500 μg of>90% pure BLIP could be isolated for every two liters of culture using this strategy. Wild-type β-lactamase and the G238S and E104K extended-spectrum mutants were expressed and purified as previously described.

BLIP Inhibition Assay. Varying concentrations of BLIP were incubated with 1 nM β-lactamase for 2 hours at 25 C. The enzyme-inhibitor incubation was conducted in 0.05 M phosphate buffer (pH 7.0) containing 1 mg/mL bovine serum albumin (BSA). Following the 2 hour incubation, cephaloridine was added at a concentration of at least 10-fold lower than the Kₘ of the β-lactamase being tested (e.g. wild-type TEM-1 β-lactamase has a Kₘ approximately 700 μM for cephaloridine, therefore 70 μM cephaloridine was added to the TEM-1/BLIP incubation). The final volume for the reaction was 0.5 mL. Hydrolysis of cephaloridine was monitored at A₂₆₀ on a Beckman DU70 spectrophotometer. The extinction coefficient used for cephaloridine was Δε=10,200 M⁻¹cm⁻¹. Plots of the concentration of free β-lactamase vs. inhibitor concentration were fit by nonlinear regression analysis to Equation 1, where Vᵢ/₀ is the fractional β-lactamase activity (steady state inhibited rate divided by the uninhibited rate), [E₀] is the total β-lactamase active site concentration, and [I₀] is the total inhibitor concentration. From the equation, apparent equilibrium dissociation constants (Kᵢ*) were determined.

Vᵢ/V₀=[E₀]+[I₀]+K₁* sqrt (([E₀]+[I₀]+Kᵢ*)2-(4[E₀][I₀])/2[E₀] (Equation 1)

### EXAMPLE 1

### Construction of a Minimal Functional BLIP Protein

BLIP is a 165 amino acid protein that is organized into two domains. The domains have a similar structure suggesting that the protein evolved by a tandem duplication of a single binding domain. Because BLIP uses both of its domains to bind a monomer of β-lactamase, each domain makes unique binding contacts. Based on the structure of the BLIP-β-lactamase co-crystal, domain 1 of BLIP appears to makes 75% of all of the interactions between the proteins.

The present inventors recognized that it would be possible to express BLIP domain 1 independent of domain 2 by truncating the protein after residue 78. The BLIP protein was truncated at residue 78 as a pG3-BLIP phage display vector. The truncation results in domain 1 being fused to the gene III protein for display on the surface of M1l13 bacteriophage. The truncation was made by PCR of the bli gene as described above for the pG3-BLIP construct except the PCR primer containing the XbaI site for fusion to gene III was designed to amplify the region ending at residue 78 rather than 165.

Phage ELISA experiments confirmed that the truncated protein is functional in β-lactamase binding. Briefly, soluble β-lactamase was used to coat the wells of a 96-well ELISA plate. The wells were blocked and phage displaying the BLIP78 truncated protein were allowed to bind the immobilized β-lactamase. The binding reactions were washed extensively, and bound phage were detected with an HRP-conjugated anti-M13 antibody. As a positive control, an equivalent number of phage that displayed the wild-type BLIP protein were also tested for binding. As a negative control, phage displaying the wild-type BLIP protein were allowed to bind a well that had been coated with BSA. The results indicated that the phage displaying BLIP78 bound β-lactamase only 6-fold less efficiently than phage displaying wild-type BLIP. The BLIP78 phage also bound the immobilized β-lactamase at levels 10-fold higher than background, suggesting that BLIP78 is functional. The weaker but detectable binding of BLIP78 to β-lactamase may result from the loss of interactions involving domain 2 or it may reflect a loss in the stability of domain 1 in the absence of domain 2 or a combination of these effects.

Next, mutants of the truncated BLIP78 that had wild-type, or better, binding to immobilized β-lactamase were isolated using the system as disclosed herein. Multiple rounds of mutagenesis and binding selection were used to evolve the BLIP78 protein back into a tight binder of β-lactamase. The directed evolution method that was employed was DNA shuffling, which produces high-frequency recombination and reassortment of mutations in sequences that have been selected for binding. This data demonstrates that it is possible to reduce the size of BLIP by multiple rounds of truncation followed by mutagenesis and selection for clones that retain inhibitory properties. Using this system, it should be possible to reduce BLIP to a cyclic peptide suitable for treatment and for use in the disruption of the interactions with small molecules in the system disclosed herein.

### EXAMPLE 2

### Altering the Binding Properties of BLIP

In order to test whether the pG3-BLIP phagemid may be used to engineer the BLIP protein for altered binding properties, two regions of BLIP were randomized. Two libraries, each randomized for three contiguous codons were constructed with randomized sequences as indicated in Tables II and III. The randomized sequences include the two loops of BLIP that are known to interact with the active site of β-lactamase and encompass residues 49-51 and 141-143. These libraries contain the Asp49 and Phe 142 residues that the inventors have shown by site-directed mutagenesis to be important for binding. Three rounds of panning on immobilized β-lactamase were performed and the sequences of binders were determined. Surprisingly, even though the Asp49 residue is critical for BLIP binding of β-lactamase, Asn was selected at position 49 much more frequently than Asp. Similarly, Phe did not predominate among the selected mutants at position 142. A strong preference for large hydrophobic amino acids like phenylalanine, however, was observed.

To determine whether the most often selected mutants in the phage display studies bind the target more tightly than wild-type BLIP, the 49-NGY-51 and 141-GIN-143 BLIP mutants were cloned and purified using the 6×His-tag as described for wild-type BLIP and the D49A and F142A mutants. The Kᵢ value for inhibition of wild-type β-lactamase was determined using an equilibrium method. The 49-NGY-51 mutant bound β-lactamase with a Kᵢ of 0.01 nM, which is approximately 10-fold tighter than binding by wild-type BLIP. These results clearly show that randomization of sections of BLIP, combined with a phage display selection, may be used to alter the binding properties of BLIP. This system may also be used to increase the binding of BLIP to other target proteins such as penicillin binding proteins. The advantage of changing the binding properties of BLIP is that the new higher affinity BLIP molecules may be used to screen for small molecule inhibitors of the protein to which BLIP binds thereby reducing or increasing its effectiveness as an inhibitor protein.

TABLE II
Loop 49-51
Round 3 Sequences

| 49-D | Y | Y-51 | |
|---|---|---|---|
| GAC | TAC | TAC | |
| ↓ | ↓ | ↓ | |
| NNS | NNS | NNS | Number of Isolates |
| N | G | Y | 8 |
| N | S | Y | 7 |
| D | Y | Y | 7 |
| N | G | N | 2 |
| N | G | H | 1 |
| E | G | W | 1 |

TABLE III
Loop 141-143
Round 3 Sequences

| 141-G | F | Y-143 | |
|---|---|---|---|
| GGG | TTC | TAC | |
| ↓ | ↓ | ↓ | |
| NNS | NNS | NNS | Number of Isolates |
| G | I | N | 3 |
| N | W | N | 2 |
| G | W | D | 2 |
| S | F | N | 1 |
| A | F | N | 1 |
| H | Y | N | 1 |
| G | F | Y | 1 |
| S | Y | S | 1 |

Screening BLIP random libraries by phage display. Residues 49-51 and 141-143 were randomized to create the L49-51 and L141-143 random libraries. Each library contained approximately 100,000 mutants. N═A,C,G,T; S═C,G. After three rounds of panning on immobilized TEM-1 β-lactamase, the sequence of BLIP from the binding mutants was determined. The amino acid sequences of the mutants are shown. The number of times a sequence was found is indicated next to each sequence.

### EXAMPLE 3

### Isolation of β-lactamase Inhibitors

The present inventors realized from the TEM-1/BLIP co-crystal that two BLIP residues, D49 and F142, mimic interactions made by Penicillin G (PenG) when bound in the active site of the β-lactamase TEM-1. To determine the importance of these two residues, the heterologous expression system described hereinabove established for BLIP in E. coli, along with site-directed mutagenesis, was used to change D49 and F142 to alanine. The inhibitory activity of both mutants was examined. It was found that both mutations decrease BLIP inhibitory activity approximately 100-fold with TEM-1 β-lactamase.

To address how these two positions effect specificity, the inhibitory activity of wild-type BLIP, as well as the D49 and. F142A mutants, was determined for two extended-spectrum β-lactamases (the G238S and the E104K TEM variants). Interestingly, the three BLIP proteins inhibited the G238S β-lactamase mutant to the same degree that they inhibited TEM-1. BLIP has a higher Kᵢ for the E104K β-lactamase mutant, suggesting that interactions between BLIP and β-lactamase residue E104 are important for wild-type levels of BLIP inhibition. Substitution of a phenylalanine at position 142 of BLIP, which interacts with the glutamic acid at position 104 of TEM-1 β-lactamase, did not substantially reduce BLIP inhibition of the E104K enzyme as observed with the TEM-1 and G238S β-lactamases. Therefore, the specific BLIP F142/β-lactamase E104 interaction appears essential for wild-type BLIP inhibitory levels.

Wild-Type BLIP Kinetics. To determine if the Histidine affected BLIP inhibitory activity, and to analyze the activity of the BLIP mutants for TEM-1, the ESBLs, and SHV-1 β-lactamase, an inhibitor assay was developed using the cephalosporin cephaloridine. Wild-type or mutant BLIP was incubated with a target for two hours after which cephaloridine (at a concentration 10-fold less than the cephaloradine Kₘ for the β-lactamase being tested) was added. Monitoring the hydrolysis of cephaloridine was used to determine the concentration of uninhibited β-lactamase. Free β-lactamase was calculated as the ratio of cephaloridine activity in the presence of a given quantity of BLIP versus cephaloridine activity in the absence of BLIP. Fitting the data obtained when incubating varying concentrations of wild-type, His-tagged BLIP with 1 nM TEM-1 β-lactamase resulted in a Kᵢ of 0.11 nM. The value returned compares favorably with the previously reported value of 0.6 nM found with BLIP purified from Streptomyces clavuligerus, and suggests that the N-terminal 6×His-tag has little effect on the binding of the inhibitor to the TEM-1 enzyme.

The affinity (Kᵢ) of wild-type BLIP was then determined for an extended spectrum β-lactamase. The G238S β-lactamase mutation is the only substitution found in TEM-19, and is also found in many extended spectrum enzymes. This single mutation increases activity for the third generation cephalosporins: ceftazidime and cefotaxime, approximately 70-fold and 40-fold, respectively. The E104K mutation, likewise, has been found in many extended-spectrum β-lactamase variants. This mutation increases the activity of β-lactamase approximately 50-fold for ceftazidime and 10-fold for cefotaxime. Wild-type BLIP was found to have a Kᵢ of 0.07 nM for G238S, and a Kᵢ of 138.5 nM for E104K. These values show that the G238S mutation has little effect on wild-type BLIP binding, while the E104K mutation interferes with binding in such a way that the Kᵢ increases 1000-fold. The fact that BLIP has binding affinity for both of these ESBLs is used in the selection system disclosed herein to screen and engineer BLIP mutants for improved binding and inhibition of these β-lactamases.

Mutant BLIP Kinetics. The crystal structure of the BLIP/TEM-1 β-lactamase inhibitory complex shows that D49 and F142 are two amino acids in the inhibitor which mimic domains of the β-lactam PenG when bound to β-lactamase. The present inventors designed the present system to target those areas of interaction for mutagenesis and for the screening of small molecules as described hereinbelow. The structural mimicry suggests that these residues maintain important interactions in the inhibitory complex. The reagents and system disclosed herein may also be used to evaluate the effect each of these amino acids has on the inhibition of TEM-1 β-lactamase and the extended-spectrum-hydrolyzing β-lactamases. The D49A and F142A mutants were used as templates for use in a system for identifying and creating novel antimicrobial agents based on the identification, isolation and selection of inhibitors that are: specific for a specific penicillin binding protein (PBP) or for a broad range of the same. The Kᵢ of each was measured with TEM-1, E104K, and G238S β-lactamases.

Both the D49A and F142A mutants demonstrated an approximate 100 to 300 fold increase in Kᵢ compared to wild-type BLIP when inhibiting TEM-1 β-lactamase. The D49A mutant inhibits TEM-1 with a Kᵢ of 8.29 nM, while the F142A mutant inhibits with a Kᵢ of 33.42 nM. The inhibitory activities of the wild-type, D49A and F142A BLIP inhibitors with the ESBLs show that the BLIP binds E104K in a different manner from that of TEM-1 and the G238S mutant. The Kᵢ values found for D49A and F142A with G238S β-lactamase were similar to the Kᵢ values found for the BLIP mutants binding TEM-1. The D49A BLIP mutant inhibited G238S with a Kᵢ of 9.35 nM. As with TEM-1, the D49A mutation reduced inhibitory activity 100-fold. Likewise, the F142A mutation reduced inhibitory activity approximately 800-fold with a Kᵢ of 54.79 nM for G238S. The fact that these two mutations in BLIP have a similar effect on the Kᵢ values for TEM-1 and G238S β-lactamases shows that BLIP inhibits G238S much in the same way in which it inhibits TEM-1. If either residue is not as important in the inhibition of G238S, then the Kᵢ value for that alanine-mutant would be closer to the wild-type BLIP Kᵢ for G238S. An example where a residue becomes less critical for inhibition of a β-lactamase mutant is position 142 with the E104K β-lactamase.

The Kᵢ of BLIP D49A with E104K is 1.51 pM, which represents a 10-fold increase from wild-type BLIP and E104K. This value suggests that BLIP residue D49 is not as critical to inhibition of E104K as it is to the other enzymes tested. In contrast to what was observed with TEM-1 and G238S, however, there was little change from the wild-type Kᵢ in the BLIP F142A mutant inhibiting E104K (Kᵢ=242.65 nM). The value for F142 does not appear nearly as important for inhibition of E104K as it is for TEM-1 and G238S.

The SHV-1 β-lactamase is 68% identical, at the amino acid level, to TEM-1 β-lactamase. How this similarity corresponds to structure is unknown as the crystal structure of SHV-1 β-lactamase has not yet been solved. Using the present invention, therefore, the lack of a crystal structure for each β-lactamase and PBP variant is overcome by isolating a functional protein. While both the TEM-1 and SHV-1 enzymes hydrolyze a similar profile of penicillins and cephalosporins, it is not clear whether the homology between the two enzymes means that BLIP should inhibit both equally well. While it may be predicted that even slight differences in the three-dimensional structure of SHV-1 compared to TEM-1 would effect BLIP binding considerably, the compositions, methods and system disclosed herein solve those problems functionally.

An additional inhibitory assay with wild-type BLIP and SHV-1 β-lactamase was performed. SHV-1 was purified to greater than 90% homogeneity (data not shown), and was bound to increasing concentrations of wild-type BLIP. The Kᵢ of BLIP for SHV-1 was found to be 991.7 nM, 9,000-fold higher than what was found for TEM-1.

Table IV summarizes the results for the wild-type BLIP and other mutants of BLIP. The inhibitory activities (Kᵢ) for wild-type BLIP and the D49A and F142A mutants with TEM-1, G238S, E104K, and SHV-1 β-lactamase are shown and expressed in nanomoles binding of β-lactamase.

TABLE IV

| BLIP | TEM-1 | G238S | E104K | SHV-1 |
|---|---|---|---|---|
| Wild-type | 0.11 ± .001 | 0.07 ± .01 | 138.5 ± 4.5 | 991.7 ± 70.2 |
| D49A | 8.29 ± .63 | 9.35 ± .83 | 1508.6 ± 52.9 | ND |
| F142A | 33.42 ± .51 | 54.79 ± 3.91 | 242.65 ± 15.7 | ND |

ND = Value not determined

As wild-type BLIP is able to bind extended-spectrum β-lactamases, the expression of this and other mutant proteins in E. coli aid in the engineering of tighter, smaller inhibitors for these β-lactamases. Protein engineering and selection systems such as the phage display system disclosed herein are easier to develop if the starting protein and target have some degree of affinity prior to the subsequent iterations of mutagenesis and screening. The present system has also been used to determine which residues are important for binding and for truncating the protein to those that encode epitopes involved in binding. As discussed hereinabove, interactions may be optimized using phage display or a comparable technique.

Determination of the Kᵢ of BLIP with SHV-1 β-lactamase shows that even though TEM-1 and SHV-1 are both class A β-lactamases, and are 68% identical, the interactions that make BLIP a tight inhibitor of TEM-1 are not conserved with SHV-1. The system of the invention allows for the determination of interactions when no crystal structure is available, as is the case for SHV-1 and most PBPs. The level of identity between TEM-1 and SHV-1 would suggests that both enzymes share a similar protein fold, however, the present inventors have used the isolation and characterization system disclosed herein to show that the assumption is not correct. The system disclosed herein, however, may be used to determine those differences that are responsible for the discrepancy in Kᵢ. The present system, for example, can be used to determine if a D104E mutation in SHV-l would improve the Kᵢ of BLIP.

### EXAMPLE 4

### Small Molecule Screen

The above described low and high affinity variants of BLIP and the phage display system described herein, may be used to perform directed molecular evolution to create variants of the BLIP protein that bind penicillin binding proteins (PBPs) and thereby kill bacteria. These variant BLIP molecules may be antibiotics. The present inventors have realized that the ideal antibiotics are small molecules. The variant BLIP proteins designed and selected for herein may be used in assays to screen for small molecules that bind to the active site of PBPs and thereby kill bacteria. Likewise, the screening system of the present invention may be used to identify molecules that disrupt the interaction between BLIP and PBPs. One example of such a system is an ELISA-based that screens for the ability of a small molecule or molecules to disrupt the interaction of BLIP and PBPs. Other means of signal detection may also be used with the present invention.

FIG. 3 depicts the positive control for the screening for a small molecule that interferes with the interaction between BLIP and PBP that uses the phage display or the other methods described hereinabove. A BLIP derivative that binds to a PBP from Staphylococcus aureus is used to find a small molecule that binds the PBP. First, the PBP is expressed and purified. Second, the purified PBP 10 is coated in wells 12, e.g., the wells 12 of a 96-well microtiter ELISA plate. Next, a purified BLIP 14 protein having a known or even an unknown affinity for PBP 10 is added to one of the wells 12. The BLIP protein 14 is then allowed to bind to the PBP 10 in the wells. Excess BLIP proteins 14 are then removed from the well by washing. Bound BLIP is detected with an anti-BLIP antibody 16 that is conjugated to an indicator, such as horseradish peroxidase for a standard ELISA assay.

FIG. 4 shows the analyte screen using the ELISA method as described in FIG. 3. Briefly, to wells 12 the BLIP protein 14 is added along with a sample of a small molecule or analyte 18 to be tested. The concentration of the analyte 18 may be titrated or varied according to its solubility, toxicity, carcinogenicity, etc. After allowing the binding reactions to occur, the wells 12 are washed repeatedly to remove any analyte 18 molecules that are not bound to the PBP 10, which was used to coat the well 12. An antibody specific for BLIP 14 is then added to each well 12 to determined if BLIP 14 remains bound to the PBP 10, and therefore, remains present in the well 12. If the small molecule or analyte 18 has affinity for the PBP 10, it will displace BLIP 14 because the concentration of affinity of the analyte 18 is higher than that of BLIP 14. The analyte 18 may interfere with the binding site of the BLIP 14 and the PBP 10. After washing, the BLIP 14 is removed from the well 12 because the analyte 18 inhibited its interaction with the PBP 10 and therefore no ELISA signal is obtained. Note that if a small molecule with no affinity for the PBP 10 were added, the result would be the same as in FIG. 3.

A polyhistidine tagged BLIP protein, as described hereinabove, may be used to facilitate the purification of BLIP. Furthermore, the HisTag may also be used to detect a signal as antibodies that recognize a poly-Histidine tag are commercially available. In operation, the screen works as follows. If the small molecule being tested is able to bind the PBP, the small molecule will displace the BLIP from the PBP and therefore the BLIP will be eliminated in the wash step. The small molecule may be able to displace the larger BLIP because, e.g., it is present in higher concentration, it has higher affinity for the PBP, and/or it blocks the binding site between the molecules. During an ELISA screen, no signal is detected where the small molecule has disrupted the interaction because BLIP does not bind or is competed-off and there is no BLIP left in the well to bind the antibody. One particular advantage to the method described herein is that the screen is easily adapted to high throughput automated robotics, as it is in a microtiter well format and the only reagent that changes from well-to-well is the small molecule being screened.

The screen disclosed here may also be used to identify small molecule inhibitors of β-lactamase itself. β-lactamase inhibitors do not themselves possess antimicrobial activity. Rather, they are used in conjunction with an older β-lactam antibiotic such as ampicillin. The inhibitor binds the β-lactamase and prevents it from inactivating ampicillin, thereby restoring the therapeutic effect of the antibiotic. Small molecule β-lactamase inhibitors such as clavulanic acid and sulbactam are widely used clinically. Since wild-type BLIP is known to bind tightly to β-lactamase, it may be used directly to screen for small molecule inhibitors of β-lactamase in the same way as described above for the PBPs.

Two studies were performed to show the use and applicability of the small-molecule screen of the present invention. In the first study, β-lactamase was used to coat the wells of a microtiter tray. Next, poly-Histidine-tagged BLIP molecules were added and allowed to bind the immobilized β-lactamase. After several rounds of washing, the BLIP molecules that remained bound were detected using an antibody that recognizes the poly-Histidine tag on BLIP. The results showed that BLIP is able to bind β-lactamase in the microtiter well.

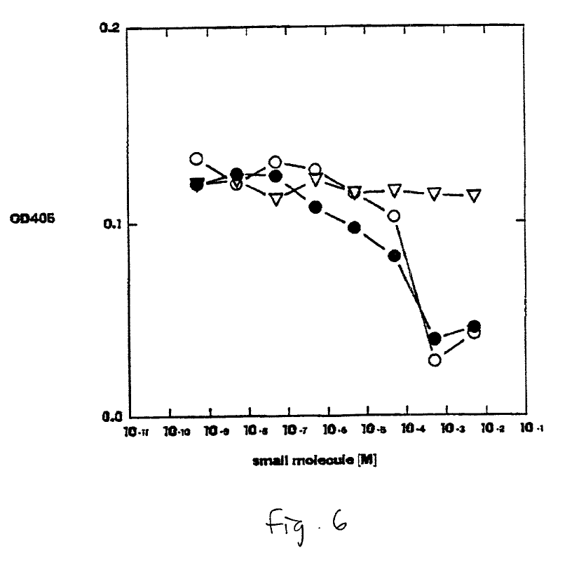

FIG. 5 is a graph that shows the results obtained during a small molecule screen for the binding of the β-lactamase active site and 6×His-tag BLIP. β-lactamase was coated on microtiter wells and incubated with poly-Histidine tagged BLIP. Increasing concentrations of the small molecule inhibitors clavulanic acid and sulbactam were added to the wells. Binding of BLIP to the immobilized β-lactamase was detected with anti-poly-Histidine monoclonal antibody conjugated to horseradish peroxidase. The product of the horseradish peroxidase is measured as OD₄₀₅ on the y-axis. As a control, the small molecule, kanamycin, which is known not to bind β-lactamase was also included. In FIG. 5, the open circles are clavulanic acid; the filled circles are sulbactam; and the open triangles are kanamycin.

The results in FIG. 5 demonstrate that a small molecule can disrupt the binding of BLIP. Increasing concentrations of the small molecule β-lactamase inhibitors sulbactam and clavulanic acid were added to the wells along with the BLIP. It was found that the small molecule inhibitors could prevent BLIP from binding β-lactamase. As a control, it was shown that the small molecule kanamycin, which is known not to bind β-lactamase, does not inhibit the binding of BLIP to β-lactamase.

FIG. 6 is a graph that shows the results from another small molecule screen using the present invention demonstrating the effect of a small molecule on the interaction between β-lactamase and BLIP fused to the surface of M13 bacteriophage. β-lactamase was coated on microtiter wells and incubated with bacteriophage that displays functional BLIP on the viral surface. Increasing concentrations of the small molecule inhibitors clavulanic acid and sulbactam were added with the bacteriophage to demonstrate the inhibitors can block BLIP binding. In FIG. 6, the open circles are clavulanic acid; the filled circles are sulbactam; and the open triangles are chloramphenicol.

The graph in FIG. 6 demonstrates that the binding of BLIP to the immobilized β lactamase was detected with anti-M13 bacteriophage monoclonal antibody conjugated to horseradish peroxidase. As a control, the small molecule, chloramphenicol, which is known not to bind β-lactamase, was also included. β-lactamase was again coated onto wells of a microtiter well tray. Phage particles that bound to β-lactamase via the BLIP-β-lactamase interaction were detected using antibody against the bacteriophage. As in the results shown in FIG. 5, increasing concentrations of the small molecule β-lactamase inhibitors clavulanic acid and sulbactam were able to prevent binding of BLIP and therefore of the bacteriophage. As a control, it was shown that the small molecule chloramphenicol, which does not bind β-lactamase, does not disrupt BLIP binding. The results from these two studies clearly show that the BLIP molecule, or engineered derivatives of the BLIP molecule, provide an efficient screen for small molecules that bind β-lactamases or PBP's.

Another way to screen for small molecules that bind PBP's or β-lactamases involves the use of radioactively labeled penicillin. The ability of the small molecule to block binding of the penicillin is read-out using a radioactive readout. Alternatively, the assay may be conducted using a fluorescent β-lactam, a β-lactamase inhibitor protein or even an antibody specific to bacteriophage antigens, thereby avoiding the need to use radioactivity.

Yet another way to screen for small molecule inhibitors or molecules that disrupt the interaction between BLIP and a BLIP target is to bind the BLIP and the BLIP target in a column, with one the molecules being tagged. Different analytes are introduced into the column one at a time and the molecule/analyte that disrupts the interaction between the proteins releases the tagged protein (that is, the one not immobilized to the column) which is detected in the effluate. A detector that is capable of detecting the presence of the tag is located downstream of the column. In this manner, a high througput, and even parallel processing, may be achieved to screen large numbers of compounds. In fact, pools of analytes may even be tested as batches, and then, when a batch shows activity, the individual components of the pool are analyzed.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method of isolating an antimicrobial agent comprising the steps of:
attaching a β-lactamase inhibitor protein target to a solid support;
exposing the β-lactamase inhibitor protein target to a β-lactamase inhibitor protein and an analyte; and
detecting the effect of the analyte on the binding of the β-lactamase inhibitor protein to its protein target, whereby a decrease in binding of the β-lactamase inhibitor protein and the β-lactamase inhibitor protein target is indicative that the analyte affects the interaction between β-lactamase inhibitor protein and its protein target.

2. The method of isolating an antimicrobial agent of claim 1, wherein the step of detecting the amount of β-lactamase inhibitor binding protein is accomplished using an enzyme-linked immunosorbent assay.

3. The method of isolating an antimicrobial agent of claim 1, wherein the step of detecting the amount of β-lactamase inhibitor binding protein is accomplished using an anti β-lactamase inhibitor binding protein conjugated to an enzyme.

4. The method of isolating an antimicrobial agent of claim 3, wherein the enzyme catalyzes a reaction that produces a chromogen.

5. The method of isolating an antimicrobial agent of claim 1, wherein the step of detecting the amount of β-lactamase inhibitor binding protein is accomplished using an anti β-lactamase inhibitor binding antibody conjugated to an radioisotope.

6. The method of isolating an antimicrobial agent of claim 1, wherein the solid support is further defined as a plate having one or more wells.

7. The method of isolating an antimicrobial agent of claim 1, wherein the solid support is further defined as a microscope slide treated to bind the β-lactamase protein target.

8. The method of isolating an antimicrobial agent of claim 1, wherein the solid support is further defined as a three dimensional matrix capable of binding the β-lactamase protein target.

9. The method of isolating an antimicrobial agent of claim 1, wherein β-lactamase inhibitor protein target is a recombinant protein.

10. The method of isolating an antimicrobial agent of claim 1, wherein the β-lactamase inhibitor protein target is a penicillin binding protein.

11. The method of isolating an antimicrobial agent of claim 1, wherein the β-lactamase inhibitor protein target is a recombinant penicillin binding protein.

12. The method of isolating an antimicrobial agent of claim 1, wherein said step of contacting the β-lactamase inhibitor binding protein and the analyte to the β-lactamase inhibitor protein target is accomplished by immobilizing the β-lactamase inhibitor protein target to oxirane beads.

13. The method of isolating an antimicrobial agent of claim 1, further comprises the steps of:
changing the side groups of the analyte based on rational drug design principles and the known structures of β-lactamase binding proteins and the target protein; and
testing the effect of the changes to the analyte using the method in accordance with claim 1.

14. The method of isolating an antimicrobial agent of claim 1, further comprising the step of exposing a bacteria to the analyte to determine if the analyte affects bacterial cell growth.

15. The method of isolating an antimicrobial agent of claim 14, wherein said step of exposing a bacterial to the analyte to determine if the analyte affects bacterial cell growth is further defined as comprising the steps of:
contacting the isolated antimicrobial agent with a bacterium; and measuring the viability of the bacterium after a predetermined period of time sufficient to determine said viability.

16. A method of isolating an antimicrobial agent comprising the steps of:
attaching a β-lactamase inhibitor protein to a solid support;
exposing the β-lactamase inhibitor protein to a β-lactamase inhibitor protein target and an analyte;
washing excess and on bound β-lactamase inhibitor protein target and analyte; and
detecting the effect of the analyte on the binding of the β-lactamase inhibitor protein target to the β-lactamase inhibitor protein by measuring the amount of bound protein target, whereby a decrease in binding between the β-lactamase inhibitor protein target and the β-lactamase inhibitor protein is indicative that the analyte affects the interaction between β-lactamase inhibitor protein and the β-lactamase inhibitor protein target.

17. The method of isolating an antimicrobial agent of claim 16, wherein the β-lactamase inhibitor protein target is a penicillin binding protein.

18. The method of isolating an antimicrobial agent of claim 16, wherein the β-lactamase inhibitor protein target is penicillin binding protein.

19. The method of isolating an antimicrobial agent of claim 16, wherein the β-lactamase inhibitor protein target is a β-lactamase.

20. A high throughput system of isolating an antimicrobial agent comprising the steps of:
attaching a recombinant β-lactamase inhibitor protein target to a solid support capable of use in a high throughput reader;
exposing the recombinant β-lactamase inhibitor protein target to a recombinant β-lactamase inhibitor protein and an analyte;
washing the solid support;
detecting the effect of the analyte on the binding of the recombinant β-lactamase inhibitor binding protein target to its protein target, wherein a decrease in binding between the recombinant β-lactamase inhibitor protein target and the recombinant β-lactamase inhibitor protein is indicative that the analyte affects the interaction between recombinant β-lactamase inhibitor protein and its protein target; and
recording the effect of the analyte on the binding of the recombinant β-lactamase inhibitor binding protein target to its protein target.

21. A system of identifying, selecting and improving an antimicrobial agent comprising the steps of:
attaching a β-lactamase inhibitor protein target to a solid support;
exposing the β-lactamase inhibitor protein target to a β-lactamase inhibitor protein and an analyte;
detecting the effect of the analyte on the binding of the β-lactamase inhibitor binding protein target to its protein target, wherein a decrease in binding between the β-lactamase inhibitor protein target and the β-lactamase inhibitor protein is indicative that the analyte affects the interaction between β-lactamase inhibitor protein and the β-lactamase inhibitor protein target;
contacting the analyte to a bacterium; and
measuring the viability of the bacterium after a predetermined period of time sufficient to determine the bacterium's viability.

22. The system of identifying, selecting and improving an antimicrobial agent of claim 21, further comprising the steps of:
changing the side groups of the analyte based on rational drug design principles and the known structures of β-lactamase binding proteins and the target protein; and
testing the effect of the changes using the method according to claim 1.
